(19) United States
(12) Patent Application Publication
(10) Pub. No.: US 2012/0294981 A1
(43) Pub. Date: Nov. 22, 2012

(54) PROCESS FOR FORMING TREATED BACKSET AND PRODUCING ETHANOL

(75) Inventors: Jeffrey B. Wicking, Ames, IA (US); Yilin Bian, Ames, IA (US); Anbin Pu, Ames, IA (US); Ping Liu, Beijing (CN)

(73) Assignee: NORTH AMERICAN PROTEIN, INC., Ames, IA (US)

(21) Appl. No.: 13/464,331

(22) Filed: May 4, 2012

Related U.S. Application Data
(60) Provisional application No. 61/488,401, filed on May 20, 2011.

(51) Int. Cl. C12N 1/14; A23L 1/29; A23K 1/16; C12P 7/06; C12P 7/10

(52) U.S. Cl. 426/61; 435/256.8; 435/256.1; 435/256.6; 435/256.7; 435/161; 435/165

(57) ABSTRACT

A process for obtaining a treated backset for use in ethanol production, the process involving treating waste material from an ethanol fermentation process with microorganisms to remove or decrease microbial cell growth inhibitors present in the waste material; and removing microbial biomass from the treated waste material to form a treated backset. The treated backset can be used in a process for producing ethanol from a fermentable biomass. Recovered microbial biomass can be used in as an animal feed or as a dietary supplement.

### CROSS REFERENCE TO RELATED APPLICATION

This application claims the benefit of U.S. Provisional Patent Application No 61/488,401 entitled ‘Process for producing ethanol’, filed on 20 May 2011, which is incorporated by reference in its entirety for all purposes.

### TECHNICAL FIELD

Methods and compositions are described for treated backset, improved processes for ethanol production using the treated backset, and animal feeds or dietary supplements obtained from the processes.

### BACKGROUND

Ethanol has become an important renewable energy source. Currently, more than 70% of the gasoline consumed in the United States is blended with ethanol. In addition, large quantities of ethanol are produced for use in beverages, medicines, and other industrial and scientific products. Almost all ethanol is produced by the fermentation and distillation of biomass, particularly grains. In the US, corn is the most widely used grain feedstock for ethanol production.

The two main industrial processes for producing ethanol are wet milling and dry grinding. The majority of ethanol plants in the US use the dry grinding process. In dry grinding, distillation is used to recover ethanol produced during microbial fermentation of the grain (whole stillage). The resulting residue following distillation consists primarily of water and non-fermentable corn solids. A centrifuge is used to separate the whole stillage to into liquid (thin stillage) and the solids (wet cake).

Current Practice in Thin Stillage ‘Disposal’

Water usage for the corn-ethanol process has been one of the criticisms of the industry. Over time, the industry has reduced its fresh water consumption per gallon of ethanol from 6:1 to 2.7:1. One of the common practices to reduce water usage is recycling approximately ⅓, but can be between 20 to 50%, of the thin stillage (backset) to the beginning of the process. The backset also provides nitrogen which is derived in part from the yeast cells that remain in the thin stillage. This type of nitrogen is beneficial for the new yeast in the subsequent ethanol fermentation, but the benefit of the nutrients only can be realized if the backset recirculation is at least 10%. Over time, the backset causes a build-up of the total solids in the process. This is because the backset also contains other components such as lactic acid, acetic acid, glycerol, cellulose, hemicelluloses and corn oil as non-fermentable solids. Such solids take up space in the processing tanks and cause an increase in viscosity which result in a reduction of ethanol production.

The total solids in the thin stillage among corn-ethanol production plants range from about 4.5% to 13%. The acids are harmful to yeast fermentation and need to be removed. The remaining thin stillage is processed by an evaporator to produce a thickened co-product called syrup. Most often, the syrup is blended back into the wet cake. After drying, the product is thus referred to as “distillers' dried grain with solubles” (DDGS). Lately, the ethanol industry has adopted a more integrated approach in that the heat from the evaporator is recovered and used in another part of the process. The majority of the DDGS is dried to 12% or less moisture content to increase the storage life.

Many fungal species are used in agriculture waste water treatment. In each waste water treatment application, a new useful product was produced, and water clean-up was achieved. However, the cleaned water is typically discharged and not reused.

The ethanol industry would benefit from: a) minimizing energy used in production of ethanol in order to maximize the net energy gain; b) minimizing negative environmental effects incident to the production process; and c) maximizing the value of co-products.

Because the backset is re-used in multiple cycles and is part of the media for yeast growth, improvement of the backset will have a profound impact on fermentation, which in turn increases ethanol yield.

### SUMMARY

A process was developed for backset “clean up” capable of removing harmful components such as acetic acid, lactic acid, and glycerol, as well as the ability to provide similar or better organic nitrogen and/or amino acids for ethanol fermentation. The process is able to produce value-added products and does not add compounds that could be potentially harmful to animal feed formed from DDGS.

In a general aspect, use of microorganisms to form a treated backset from the waste material of an ethanol fermentation process, and use of the treated backset to produce ethanol are disclosed.

A first aspect, a process for obtaining a treated backset for use in ethanol production includes:

(a) treating waste material from an ethanol fermentation process with microorganisms to remove or decrease microbial cell growth inhibitors present in the waste material; and

(b) removing microbial biomass from the treated waste material to form a treated backset.

The waste material can originate from any ethanol fermentation process involving fermentation and distillation of a suitable fermentable biomass. Suitable fermentable biomass includes corn, milo, other grains, or enzyme or microbial treated cellulosic material.

Preferably, the fermentation process is a dry grinding process for producing ethanol from grain. Preferably, the waste material is thin stillage separated from whole stillage collected after fermentation and distillation of the grain. A suitable grain is corn.

Suitable microorganisms include a fungus, for example, a fungus selected from Aspergillus spp, Rhizopus spp, Trichoderma spp, or Rhizomucor spp.

In one preferred embodiment, the fungus is an Aspergillus spp. More preferably, the Aspergillus spp is Aspergillus oryzae. var, (A.o) which is particularly suitable for the methods disclosed herein.

The waste material may be treated by incubation with a fungus in a fermentation vessel. The incubation preferably involves a culture vessel having fungal culture medium, fungi, and the waste material. An inoculation of the waste material may be achieved by the use of fungal spores. The incubation may be a batch process or involve a continuous feed system.

Preferably, the incubation is a liquid fermentation process. More preferably, the incubation is a liquid fermentation process with thin stillage. The period of time of incubation may by approximately 24 to 60 hours. Preferably, the period of time is approximately 48 hours. It will be appreciated that the time of incubation can be longer or shorter, depending on the type of material being treated, the volume of material, and the microorganism being used.

Preferably, the treated backset contains microbial metabolites suitable for yeast fermentation of feedstock such as grain, to form alcohol. The treated backset has reduced yeast cell growth inhibitors when compared to un-treated waste material from an ethanol fermentation process. The yeast cell growth inhibitors include acetate, lactic acid, nitrogen, glycerol, and total COD.

The microbial biomass recovered may be used as an animal feed or dietary supplement.

A second aspect, is that a treated backset is used in the production of ethanol according to the first aspect.

Treated backset formed from an ethanol fermentation process can be reused in a subsequent ethanol fermentation process to improve yields of ethanol.

A third aspect, a process for producing ethanol by fermentation,includes:

(a) providing a fermentable biomass;

(b) adding treated backset to the fermentable biomass,

(c) carrying out fermentation of the fermentable biomass and the treated backset to form ethanol; and

(d) recovering the ethanol.

Preferably, the fermentable biomass is corn, milo, other grains, or enzyme or microbial treated cellulosic material. More preferably, the biomass is corn.

The waste material is typically thin stillage.

Preferably, the treated backset is produced according to the first aspect.

In a preferred embodiment, the treated backset is added to the process where the untreated backset would usually be added. Therefore, no alternation is necessary to the ethanol production process. The quantity of the treated backset in total process water may be 20% treated backset and 80% water, or 30% treated backset and 70% water, or 40% treated backset and 60% water, or 50% treated backset and 50% water.

Preferably, the waste material contains about 5-20% total solids before the microbial treatment. More preferably, if the solids content is higher than about 8%, the total solids is adjusted to less than about 8% before the microbial treatment.

The pH of the backset may be adjusted to be in the range of 4 to 6.

The pH may be adjusted by diluted sodium hydroxide or diluted sulfuric acid or hydrochloric acid. Preferably, the pH is between 4 to 4.5.

In a preferred embodiment, the waste material is converted to treated backset and the treated backset is used in a subsequent ethanol production process batch.

A fourth aspect, a process for producing ethanol by fermentation is provided including:

(a) providing a fermentable biomass;

(b) adding treated backset to the fermentable biomass,

(c) carrying out fermentation of the fermentable biomass and treated backset to form ethanol;

(d) recovering the ethanol and forming waste material;

(e) treating the waste material by microorganisms to remove or decrease microbial cell growth inhibitors present in the waste material;

(f) removing microbial biomass from the treated waste material to form a treated backset;

(g) adding the treated backset to a fermentable biomass (cycling back to step (b) and repeating the process to form ethanol);

(h) carrying out fermentation of the fermentable biomass and the treated backset to form ethanol; and

(i) recovering the ethanol.

Preferably, the treated backset is produced according to the first aspect.

In a fifth aspect, biomass obtained from forming a treated backset is used in an animal feed or supplement.

Preferably, the biomass is obtained as a by-product of the process according to the first aspect.

In a sixth aspect, an animal feed or supplement includes biomass obtained from the microbial process that forms a treated backset.

Throughout this specification, unless the context requires otherwise, the word “comprise”, or variations such as “comprises” or “comprising”, and “includes” or “including” will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is solely for the purpose of providing a context for the present invention. It is not to be taken as an admission that any or all of these matters form a component of the prior art base or were common general knowledge in the field relevant to the present invention as it existed prior to development of the present invention.

In order that the present invention may be more clearly understood, preferred embodiments will be described with reference to the following drawings and examples.

### BRIEF DESCRIPTION OF THE DRAWINGS

FIG. 1 shows a schematic of microbial treatment of backset and generation of clarified thin stillage (CTS) according to the present invention.

FIG. 2 shows reduction of acids in thin stillage by fungal fermentation.

FIG. 3 shows reduction of glycerol in thin stillage by fungal fermentation.

FIG. 4 sets out a laboratory procedure for testing treated backset in ethanol production, modified from industrial procedures.

### DETAILED DESCRIPTION

### Mode(s) for Carrying Out the Invention

### Selection of Fungal Species for Backset Treatment

Fungal Aspergillus oryzae, var, (A.o) was selected for the backset cleanup. A.o has been used for soy sauce and rice wine production for over 1000 years and is approved for use as a Direct Fed Microbial (DFM) by both the Food and Drug Administration (FDA) and Association of American Feed Control Officials (AAFCO). After careful screening, a fast growing subculture of A.o was selected to shorten the treatment time. Other fungal species, such as Aspergillus niger, var, Rhizopus niveus, Rhizopus oryzae, var, Trichoderma longibrachiatum (formerly, reesei), and Rhizomucor (Mucor-) miehei can also be used.

### Backset Treatment Using a Fungal Fermentation Process

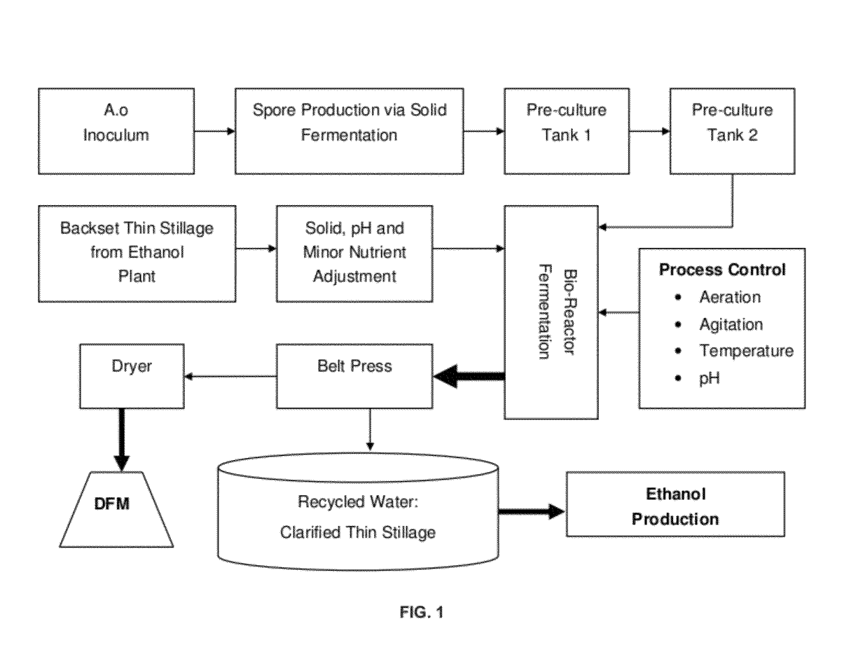

A novel process for the backset treatment using A.o has been developed. The technology includes the growth of fungal spores on an industrial scale using solid fermentation methods and pre-culture cultivation using backset as media. The pre-culture tank size was 1-10% of the bio-reactor used for treated backset production. Fermentation conditions and bio-reactor design were calculated to provide optimal aeration and agitation for fungal growth (FIG. 1).

The majority of corn-ethanol production uses a batch process which generates thin stillage every 60-72 hours. In contrast, one method disclosed herein was designed as a batch system of which the backset in the bio-reactor was optimized to 48 hours.

### Using Treated Backset in Ethanol Fermentation Process

The composition of a treated backset as compared with the backset is shown in Table 1. In this case, a backset with a solid of 4% was used. After a 48-hour A. o treatment, reduction of acetate from the backset was the most significant, followed by total COD, nitrogen and lactic acid. Total ash and glycerol had only a minor reduction.

TABLE 1
Composition of the backset after 48-hour
A.o treatment as treated backset

| | | Treated | |
|---|---|---|---|
| | Backset, % | Backset, % | Reduction, % |
| Total Solid | 4.00 | 2.06 | 48.5 |
| COD (chemical oxygen | 63000 | 36400 | 42.2 |
| demand) | | | |
| Total Nitrogen (as is | 0.215 | 0.148 | 31.2 |
| based) | | | |
| Ash (as is based) | 0.367 | 0.357 | 2.7 |
| Acetate | 0.40 | 0.09 | 77.5 |
| Lactic acid | 1.22 | 0.85 | 30.3 |
| Glycerol | 1.032 | 0.999 | 3.2 |

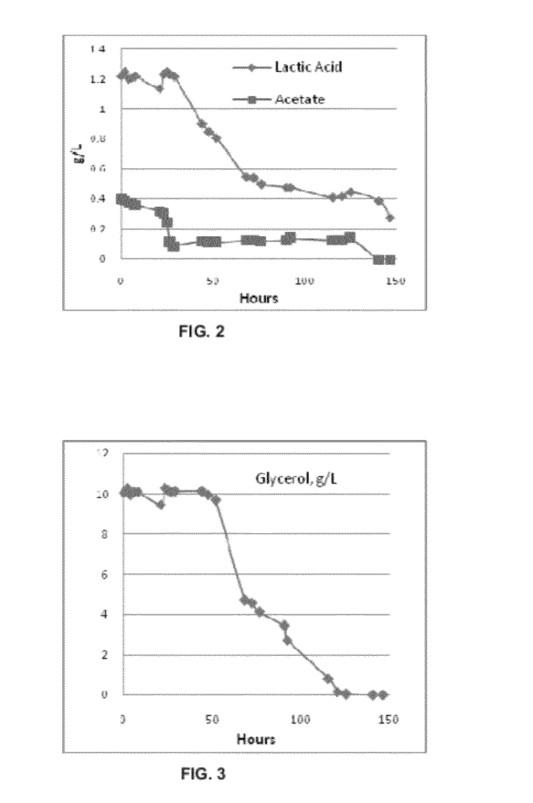

The reduction of nitrogen in general would be viewed as a negative effect, but removal of acids can reduce stress on the yeast so it is considered to be more beneficial to the subsequent ethanol production. Therefore, the use of treated backset as the improved ‘backset’ was evaluated for production of ethanol from dry grind corn. The fungal treatment reduces total solids in the backset by between 40 to 70%, depending on the type and source of the backset. The fungus consumes acetate, then lactic acid and lastly, glycerol. Therefore, the reduction in acetate is up to 77%. Prolonging the fermentation time could further remove lactic acid (FIG. 2) and glycerol (FIG. 3).

### EXAMPLES

The examples presented herein are exemplary and not meant to limit the invention.

### Example 1

Fresh backset thin stillage at 7.7% solids was obtained from a corn-ethanol production plant and diluted with fresh water to a total solid content of 6%. The backset was treated by autoclave at 121° C. to eliminate or reduce possible microbial contaminants, such as wild yeast and lactic acid bacteria. In an actual large scale production setting, the incoming fresh backset is piped directly from the centrifuge or the thin stillage central holding tank through a heat exchange system before entering the bio-reactor. The backset was adjusted to pH 4-4.5 and cooled to temperature 25-30° C. A.o preculture (or other suitable fungi preculture) at volume ratio of 1-10% was added to the bio-reactor. Sterilized compressed air was pumped into the bio-reactor, a 14-L bio-reactor, at 0.1 to 1.0 vvm (volume per volume per minute), and the total content was mixed at 100-600 rpm. For the next 48 hours, the temperature of the bio-reactor was kept at 25-30° C.

After fermentation, the total content from the bio-reactor was emptied onto a filter with a mesh size smaller than about 40-mesh (US). The fungal cells were collected from the top of the screen and a belt-press or roller-press was used to dewater as much as possible. The use of selected flocculent chemicals with GRAS (Generally Recognised as Safe) status can be used to improve the dewatering process. The filtrate liquid was combined with liquid from the dewatering process to form the treated backset to replace the backset. The treated backset was heated to at least about 80° C. to kill the A.o before being used in ethanol production. In the actual large scale production, the treated backset could go through a heat exchanger before being mixed in with the corn slurry.

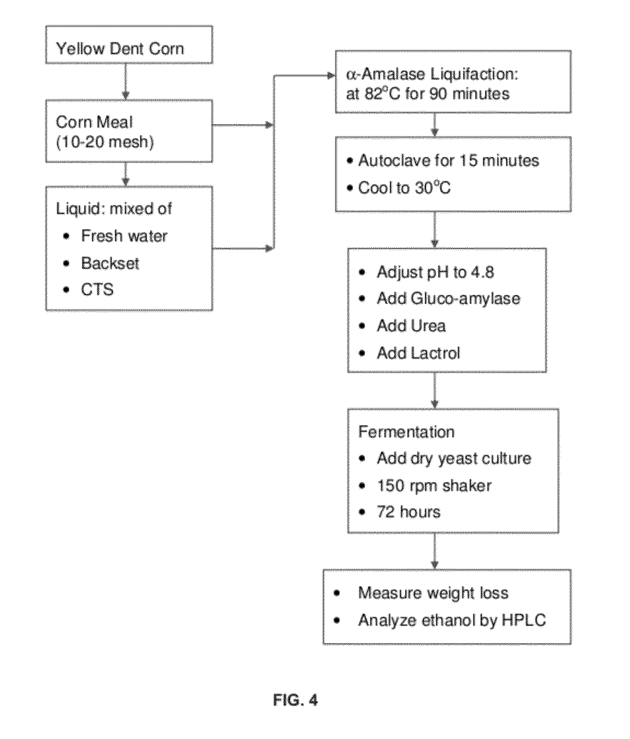

The treated backset with a total solid of 3.0% was compared to the original backset that had a solids content of 7.7%. The diagram of the laboratory fermentation procedure was modified from an industrial procedure and is shown in FIG. 4. After the fermentation, High Performance Liquid Chromatography (HPLC) was used to determine the ethanol concentration in the ‘beer’.

The results shown in Table 2 concluded that the ethanol yield using treated backset was increased by 3.69% and 5.67% with 30% treated backset and 50% treated backset respectively as compared with the original backset. The reduction in overall solid contents and portions of organic acids by using treated backset helped the yield increase. Treated backset also provided organic nitrogen in the form of fungal cells to the yeast fermentation, therefore yields from a 50% treated backset was greater than 30% treated backset. In practice, it is not acceptable or economical to use all fresh water in commercial production of ethanol.

TABLE 2
Effect of treated backset on ethanol yield in laboratory

| | Ethanol | | Yield |
|---|---|---|---|
| Liquid make up | Concentration, | Ethanol Yield, | Increase to |
| in corn mesh | g/L | gallon/bushel* | Control, % |
| 100% Fresh water | 99.90 | 2.625 | 10.20 |
| 30% treated backset + | 99.23 | 2.470 | 3.69 |
| 70% Fresh Water | | | |
| 50% treated backset + | 97.73 | 2.517 | 5.67 |
| 50% Fresh Water | | | |
| 30% Backset + 70% | 94.63 | 2.382 | — |
| Fresh Water (Control) | | | |

1 bushel = 56 lbs of yellow dent corn

### Example 2

The treated backset was evaluated at a laboratory located at an ethanol production facility using a typical industrial process. The basic ethanol fermentation steps were similar to the method described in FIG. 4, but particle size of the ground corn and some of the additives were adjusted to the particular manufacturer. However, the liquid make-up of the corn mesh was different from that in the Example 1. The ‘Process Water’, not fresh water, was used with the backset. The process water included evaporation condensate, dryer condensate and beer degas vent condensate. A small amount of fresh water plus backset was used to make up the final volume of the liquid. The corn solid in mesh was about 15%. This ethanol plant uses about 20% thin stillage as backset. In Table 3, treated backset or untreated backset was mixed with other process water at a 20% to 80% ratio. The backset from this plant had 10% solids and the treated backset used had 2% solids. After fermentation, ethanol yield from treated backset treatment had a 2.15% gain relative to the backset treatment. The HPLC analysis of the thin stillage also showed that using a 20% treated backset at the beginning of the process reduced the residue total sugar, organic acids, acetate and glycerol in newly generated thin stillage which indicated improvement in yeast performance of converting sugar to ethanol. Therefore, improving the quality of the backset increased the efficiency of the ethanol fermentation process (Table 4). Over time, with many cycles of fungal treatment and treated backset reuse, the efficiency of ethanol fermentation could be even higher. It was estimated that for this ethanol plant of 50 MGY (million gallons per year), with a 2.15% yield gain and a price of ethanol at $2.00/gal, there would be extra income of $2.15 million dollars per year without modification of their current process.

TABLE 3
Effect of treated backset on Ethanol Yield Using
a Typical Industrial Process

| | Ethanol | Ethanol | Yield |
|---|---|---|---|
| | Concentration, | Yield, | Increase to |
| Liquid make up in corn mesh | g/L | Gal/bu | Control, % |
| 20% backset + 80% Process | 125.6 | 3.038 | — |
| Water | | | |
| 20% treated backset + 80% | 128.3 | 3.103 | 2.15 |
| Process Water | | | |

TABLE 4
Effects of treated backset on Chemical Residuals
in Newly Generated Thin Stillage

| | | 20% treated | |
|---|---|---|---|
| Chemicals Residuals | 20% Backset | backset | Change, % |
| Total Residual Sugar, | 29.14 | 24.18 | −17.02 |
| g/L | | | |
| Lactic Acid, g/L | 1.355 | 1.083 | −20.07 |
| Succinic Acid, g/L | 1.363 | 1.023 | −24.94 |
| Acetic Acid, g/L | 0.535 | 0.488 | −8.79 |
| Glycerol, g/L | 11.53 | 10.14 | −12.06 |
| Solid, % | 10.03 | 5.58 | −44.37 |
| pH | 4.37 | 4.72 | +8.01 |

Fungal Biomass Composition as Compared to Yeast Products in Animal Feed Supplement Applications

In addition to ethanol yield improvement by the treated backset, the fungal A.o is a known microorganism in feed and has food uses. Direct fed microbial (DFM) has a long history as a feed supplement in animal feed. The microorganism cell mass or extract would be dried to a powder or a small particle size. For example, yeast products derived from Saccharomyces cerevisiae such as Yea-Sacc™ 1026 (Alltech, Inc. USA), XP™ (Diamond V Mills, Inc. USA) and Biomate P1us™ (Chr. Hansen Co, USA) have been the subject of more than 100 studies related to the impact on rumen fermentation and performance. Studies of A.o products have resulted in over 28 publications. The FDA and AAFCO have approved the use of yeast and A.o in feed products. DFMs effectiveness is usually apparent in the following situations:

Young animals

Weaning or dietary changes

Periods of stress

Antibiotic therapy

After the A.o fermentation in the bio-reactor, the A.o cell mass was harvested, dried, and ground to a product which can be used directly or mixed with other feed ingredients for animals. Table 5 lists the nutritional composition and key amino acid composition in the A.o cell mass. Because it is produced in thin stillage it also contains corn oil, protein, and fiber which make it a unique feed product as compared to the same A.o grown in another media.

TABLE 5
Composition of A.o. Product (dry based)

| | |
|---|---|
| %, CRUDE PROTEIN | 37-42 |
| % CRUDE FAT | 10-28 |
| % CRUDE FIBER | 19 |
| % ASH | 5-10 |
| % MOISTURE | 5-10 |
| COLOR | LIGHT YELLOW |
| BULK DENSITY, LB/FT³ | 28-30 |

Besides the nutritional composition, there is no published data in respect to the complex carbohydrate composition and linkage (Table 6). The resultant tests indicated that 1,3 and 1,4 glucan linkages were the predominate linkages with 31.4% and 27.7% respectively of the total carbohydrate linkages. It was also the first time that the precise content of mannoligoscchride (MOS) in A.o was evidenced. The effect of both 1,3 linked glucan and MOS content in yeast based feed supplements has been studied by many researchers and companies. It was not previously known that the A.o cell mass can provide a significant amount of both glucan and MOS. The glucan and MOS content can be further purified or concentrated by either chemical or enzymatic methods to produce human supplements.

TABLE 6
Composition of Glucan and MOS in A.o

| | |
|---|---|
| % 1,3 glucan | 31.4 |
| % 1,4 glucan | 27.7 |
| % MOS | 16 |
| % 1,6 glucan | 0.6 |
| % Total Carbohydrates | 61 |

### Example 3

### Antimicrobial Activity of A.o Extract

The use of A.o as a probiotic in poultry has been reported but the previous research did not demonstrate the direct linkage of A.o as an antimicrobial agent. It was unknown at the time that A.o contained glucan and MOS and both could directly improve the gastrointestinal tract in animals. Antimicrobial activity of A.o grown in thin stillage was evaluated using disk inhibition zone assays. A.o mass was harvested by a 100 mesh screen. Ultrasonic's was applied to break up the cell wall and the content was filtered and centrifuged. A 10 μl of supernatant was filtered again under sterile conditions before being added to a 6 mm filter paper disk. The disk was placed on SMAC agar plate of E. coli 0157:H7 which had been previously spread with 10⁵ CFUs/ml. Multiple plates were repeated and incubated at 37° C. for 48 hours. The thickness (mm) of the inhibition zone around the disk (colony-free-perimeter) was measured to determine the effectiveness of the A.o extract. The extracts from A.o grown in modified thin stillage had shown improvement in antimicrobial activity, especially when thin stillage pH was adjusted from 4 to above 5 (Table 7). This data demonstrates that A.o can act as an antimicrobial agent and is capable of being further optimized. This has not previously been recorded in the literature.

TABLE 7
E. coli inhibition zones produced by A.o extracts

| A.o Growth Media | Inhibition Zone, mm |
|---|---|
| Original thin stillage, pH = 4 | 3.38 |
| Thin stillage at pH 5.5 | 5.50 |
| Thin stillage modified with 0.1% molasses | 3.75 |
| Thin stillage modified with 0.25 ammonia sulfate | 3.50 |

### Example 4

### Immune System Enhancement of A.o Extract

A.o mass was harvested by a 100 mesh screen. Ultrasonic treatment was applied to break up the cell walls and the content was filtered and centrifuged. The extract was tested using MTT colorimetric assay for lymphocyte proliferation with chicken splenocytes. In this method, an increase in absorbance relates to an increase in cell numbers and therefore measures the cell growth in response to A.o extract. The thin stillage contains some yeast cell extract, as a result, it had some absorbance. The A.o extracts obtained from the fungi grown in different thin stillage showed variation in the absorbance but, overall, the extract had positive improvement on the spleen cell growth (Table 8).

TABLE 8
The Effect of A.o Extract on Chicken Splenocytes

| | MTT Assay, |
|---|---|
| Treatments | absorbance at 600 nm |
| Purified thin stillage | 0.1577 |
| Extract from A.o grown in thin stillage at pH 4 | 0.2229-0.449 |
| Extract from A.o grown in thin stillage at pH 5.5 | 0.304 |
| Extract from A.o grown in thin stillage with | 0.241 |
| 0.05% molasses | |

Although purified thin stillage has some effect, (probably due to residual yeast cells), the addition of A.o resulted in increased absorbance levels. As with the previous result this effect is clearly capable of further optimization.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

We claim:

1. A process for obtaining a treated backset for use in ethanol production, the process comprising:
(a) treating waste material from an ethanol fermentation process with microorganisms to remove or decrease microbial cell growth inhibitors present in the waste material; and
(b) removing microbial biomass from the treated waste material to form a treated backset.

2. The process according to claim 1 wherein the waste material is thin stillage.

3. The process according to claim 1 wherein the microorganisms are fungus.

4. The process according to claim 3 wherein the fungus is selected from the group consisting of Aspergillus spp, Rhizopus spp, Trichoderma spp, and Rhizomucor spp.

5. The process according to claim 4 wherein the fungus is an Aspergillus spp.

6. The process according to claim 5 wherein the fungus is Aspergillus oryzae.

7. The process according to claim 6 wherein the fungus is Aspergillus oryzae, var, (A.o).

8. The process according to claim 1 wherein the waste material contains 5-20% total solids before microbial treatment.

9. The process according to claim 8 wherein the total solids is less than 8% before the microbial treatment.

10. The process according to claim 1 wherein pH of the backset is adjusted to be in the range of 4 to 6.

11. The process according to claim 10 wherein the pH is adjusted by selected from a group consisting of diluted sodium hydroxide, diluted sulfuric acid, and hydrochloric acid to a pH between 4 to 4.5.

12. The process according to claim 1 further comprising recovering the microbial biomass for use as an animal feed or dietary supplement.

13. A process for producing ethanol by fermentation, the process comprising:
(a) providing a fermentable biomass;
(b) adding treated backset produced by the process according to claim 1 to the fermentable biomass;
(c) carrying out fermentation of the fermentable biomass and the treated backset to form ethanol; and
(d) recovering the ethanol.

14. The process according to claim 13 wherein the fermentable biomass is selected from a group consisting of corn, milo, other grains, enzyme treated cellulosic material, microbial treated cellulosic material, and combustion thereof.

15. The process according to claim 14 wherein the fermentable biomass is corn.

16. A process for producing ethanol by fermentation, the process comprising:
(a) providing a fermentable biomass;
(b) adding treated backset obtained from the process according to claim 1 to the fermentable biomass;
(c) carrying out fermentation of the fermentable biomass and treated backset to form ethanol;
(d) recovering the ethanol and forming waste material;
(e) treating the waste material by microorganisms to remove or decrease microbial cell growth inhibitors present in the waste material;
(f) removing microbial biomass from the treated waste material to form a treated backset;
(g) adding the treated backset to a fermentable biomass;
(h) carrying out fermentation of the fermentable biomass and the treated backset to form ethanol; and
(i) recovering the ethanol.

17. The process according to claim 16 wherein the fermentable biomass is selected from the group consisting of corn, milo, other grains, enzyme treated cellulosic material and microbial treated cellulosic material.

18. The process according to claim 17 wherein the fermentable biomass is corn.

19. The process according to claim 16 wherein the microorganisms are fungus.

20. The process according to claim 19 wherein the fungus is selected from the group consisting of Aspergillus spp, Rhizopus spp, Trichoderma spp, and Rhizomucor spp.

21. The process according to claim 20 wherein the fungus is an Aspergillus spp.

22. The process according to claim 21 wherein the fungus is Aspergillus oryzae.

23. The process according to claim 22 wherein the fungus is Aspergillus oryzae, var, (A.o).

24. The process according to claim 16 further comprising recovering the microbial biomass for use as an animal feed or dietary supplement.

25. An animal feed or dietary supplement comprising microbial biomass obtained from a microbial process according to claim 1 to form a treated backset.
